SEQUENCE LISTING (22 sequences)

SEQ ID NO: 1 - protein, 269 aa, Thermomyces lanuginosus [sequence omitted]

SEQ ID NO: 2 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNAIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCS

SEQ ID NO: 3 - DNA, 31 bp, Artificial Sequence
  Primer
ttgaattgaaaatagattgatttaaaacttc

SEQ ID NO: 4 - DNA, 25 bp, Artificial Sequence
  Primer
ttgcatgcgtaatcatggtcatagc

SEQ ID NO: 5 - DNA, 26 bp, Artificial Sequence
  Primer
ttgaattcatgggtaataactgatat

SEQ ID NO: 6 - DNA, 32 bp, Artificial Sequence
  Primer
aaatcaatctattttcaattcaattcatcatt

SEQ ID NO: 7 - DNA, 11 bp, Artificial Sequence
  Primer
gtactaaaacc

SEQ ID NO: 8 - DNA, 11 bp, Artificial Sequence
  Primer
ccgttaaattt

SEQ ID NO: 9 - DNA, 45 bp, Artificial Sequence
  Primer
ggatgctgttgactccggaaatttaacggtttggtcttgcatccc

SEQ ID NO: 10 - DNA, 14 bp, Artificial Sequence
  Primer
atgcaatttaaact

SEQ ID NO: 11 - DNA, 14 bp, Artificial Sequence
  Primer
cggcaatttaacgg

SEQ ID NO: 12 - DNA, 44 bp, Artificial Sequence
  Primer
ggtattgtcctgcagacggcaatttaacggcttctgcgaatcgc

SEQ ID NO: 13 - DNA, 26 bp, Artificial Sequence
  Primer
tctgtgaggcctatggatctcagaac

SEQ ID NO: 14 - DNA, 27 bp, Artificial Sequence
  Primer
gatgctgcatgcacaactgcacctcag

SEQ ID NO: 15 - DNA, 59 bp, Artificial Sequence
  Primer
cctctagatctcgagctcggtcaccggtggcctccgcggccgctggatccccagttgtg

SEQ ID NO: 16 - DNA, 33 bp, Artificial Sequence
  Primer
gcaagcgcgcgcaatacatggtgttttgatcat

SEQ ID NO: 17 - DNA, 30 bp, Artificial Sequence
  Primer
gaatgacttggttgacgcgtcaccagtcac

SEQ ID NO: 18 - DNA, 25 bp, Artificial Sequence
  Primer
cttattagtaggttggtacttcgag

SEQ ID NO: 19 - DNA, 37 bp, Artificial Sequence
  Primer
gtccccagagtagtgtcactatgtcgaggcagttaag

SEQ ID NO: 20 - DNA, 64 bp, Artificial Sequence
  Primer
gtatgtcccttgacaatgcgatgtatcacatgatataattactagcaagggaagccgtgcttgg

SEQ ID NO: 21 - DNA, 24 bp, Artificial Sequence
  Primer
ctcccttctctgaacaataaaccc

SEQ ID NO: 22 - DNA, 66 bp, Artificial Sequence
  Primer
cctctagatctcgagctcggtcaccggtggcctccgcggccgctgcgccaggtgtcagtcaccctc

(19) United States
(12) Patent Application Publication
(10) Pub. No.: US 2004/0152180 A1
(43) Pub. Date: Aug. 5, 2004

(54) Lipolytic enzyme variant

(76) Inventors: Steffan Minning, Frederiksberg C (DK); Jesper Vind, Vaerlose (DK); Sanne O. Schroder Glad, Ballerup (DK); Steffen Danielsen, Kobenhavn O (DK); Kim Borch, Birkerod (DK)

Correspondence Address: NOVOZYMES NORTH AMERICA, INC., 500 FIFTH AVENUE, SUITE 1600, NEW YORK, NY, 10110, US

(21) Appl. No.: 10/250,522

(22) PCT Filed: Jan. 10, 2002
(86) PCT No.: PCT/DK02/00016

(30) Foreign Application Priority Data
Jan. 10, 2001 (DK) PA 2001 00032

(51) Int. Cl. C12N 9/16

(52) U.S. Cl. 435/196; 435/198

(57) ABSTRACT

Novel lipolytic enzymes are disclosed which are capable of removing substantial amounts of lard from a lard stained swatch in a one cycle wash. Preferred lipolytic enzymes are variants of the Humicola lanuginosa lipase which may be prepared by recombinant DNA techniques. The enzymes are advantageously used in detergent compositions.

### FIELD OF THE INVENTION

The present invention relates to variants of fungal lipolytic enzymes, particularly variants with improved thermostability, and to methods of producing and using such variants.

### BACKGROUND OF THE INVENTION

It is known to use fungal lipolytic enzymes, e.g. the lipase from Thermomyces lanuginosus (synonym Humicola lanuginosa), for various industrial purposes, e.g. to improve the efficiency of detergents and to eliminate pitch problems in pulp and paper production. In some situations, a lipolytic enzyme with improved thermostability is desirable (EP 374700, WO 9213130).

WO 92/05249, WO 92/19726 and WO 97/07202 disclose variants of the T. lanuginosus (H. lanuginosa) lipase.

### SUMMARY OF THE INVENTION

The inventors have found that the thermostability of a fungal lipolytic enzyme can be improved by certain specified substitutions in the amino acid sequence.

Accordingly, the invention provides a variant of a parent fungal lipolytic enzyme, which variant comprises substitution of one or more specified amino acid residues and is more thermostable than the parent lipolytic enzyme. The invention also provides a method of producing a lipolytic enzyme variant comprising:

a) selecting a parent fungal lipolytic enzyme,

b) in the parent lipolytic enzyme substituting at least one specified amino acid residue,

c) optionally, substituting one or more amino acids other than b),

d) preparing the variant resulting from steps a)-c),

e) testing the thermostability of the variant,

f) selecting a variant having an increased thermostability, and

g) producing the selected variant.

The specified amino acid residues comprise amino acid residues corresponding to any of 21, 27, 29, 32, 34-42, 51, 54, 76, 84, 90-97, 101, 105, 111, 118, 125, 131, 135, 137, 162, 187, 189, 206-212, 216, 224-234, 242-252 and 256 of SEQ ID NO: 1.

The thermostability may particularly be increased by more than 4° C. The substitutions may be with a different amino acid residue, particularly one different from Pro.

### DETAILED DESCRIPTION OF THE INVENTION

Parent Lipolytic Enzyme

The lipolytic enzyme to be used in the present invention is classified in EC 3.1.1 Carboxylic Ester Hydrolases according to Enzyme Nomenclature (available at http://www.chem.qmw.ac.uk/iubmb/enzyme). The substrate specificity may include activities such as EC 3.1.1.3 triacylglycerol lipase, EC 3.1.1.4 phospholipase A2, EC 3.1.1.5 lysophospholipase, EC 3.1.1.26 galactolipase, EC 3.1.1.32 phospholipase A1. EC 3.1.1.73 feruloyl esterase.

The parent lipolytic enzyme is fungal and has an amino acid sequence that can be aligned with SEQ ID NO: 1 which is the amino acid sequence shown in positions 1-269 of SEQ ID NO: 2 of U.S. Pat. No. 5,869,438 for the lipase from Thermomyces lanuginosus (synonym Humicola lanuginosa), described in EP 258 068 and EP 305 216. The parent lipolytic enzyme may particularly have an amino acid sequence with at least 50% homology with SEQ ID NO: 1. In addition to the lipase from T. lanuginosus, other examples are a lipase from Penicillium camembertii (P25234), lipase/phospholipase from Fusarium oxysporum (EP 130064, WO 98/26057), lipase from F. heterosporum (R87979), lysophospholipase from Aspergillus foetidus (W33009), phospholipase A1 from A. oryzae (JP-A 10-155493), lipase from A. oryzae (D85895), lipase/ferulic acid esterase from A. niger (Y09330), lipase/ferulic acid esterase from A. tubingensis (Y09331), lipase from A. tubingensis (WO 98/45453), lysophospholipase from A. niger (WO 98/31790), lipase from F. solanii having an isoelectric point of 6.9 and an apparent molecular weight of 30 kDa (WO 96/18729).

Other examples are the Zygomycetes family of lipases comprising lipases having at least 50% homology with the lipase of Rhizomucor miehei (P19515) having the sequence shown in SEQ ID NO: 2. This family also includes the lipases from Absidia reflexa, A. sporophora, A. corymbifera, A. blakesleeana, A. griseola (all described in WO 96/13578 and WO 97/27276) and Rhizopus oryzae (P21811). Numbers in parentheses indicate publication or accession to the EMBL, GenBank, GeneSeqp or Swiss-Prot databases.

Amino Acid Substitutions

The lipolytic enzyme variant of the invention comprises one or more substitutions of an amino acid residue in any of the regions described above. The substitution may, e.g., be made in any of the regions corresponding to 206-208, 224-228, 227-228, 227-231, 242-243 and 245-252 of SEQ ID NO: 1. The amino acid residue to be substituted may correspond to residue Y21, D27, P29, T32, A40, F51, S54, I76, R84, I90, G91, N94, N101, S105, D111, R118, R125, A131, H135, D137, N162, V187, T189, E210, G212, S216, G225, L227, I238 or P256 of SEQ ID NO: 1. Some particular substitutions of interest are those corresponding to D27N/R/S, P29S, T32S, F51I/L, I76V, R84C, I90L/V, G91A/N/S/T/W, L93F, N94K/R/S, F95I, D96G/N, N101D, D111A/G, R118M, A131V, H135Y, D137N, N162R, V187I, F211Y, S216P, S224I/Y, G225P, T226N, L227F/P/G/V, L227X, V228C/I, 238V and P256T of SEQ ID NO: 1.

The total number of substitutions in the above regions is typically not more than 10, e.g. one, two, three, four, five, six, seven or eight of said substitutions. In addition, the lipolytic enzyme variant of the invention may optionally include other modifications of the parent enzyme, typically not more than 10, e.g. not more than 5 such modifications. The variant may particularly have a total of not more than 10 amino acid modifications (particularly substitutions) compared to the parent lipolytic enzyme. The variant generally has a homology with the parent lipolytic enzyme of at least 80%, e.g. at least 85%, typically at least 90% or at least 95%.

Lipolytic Enzyme Variant

The variant has lipolytic enzyme activity, i.e. it is capable of hydrolyzing carboxylic ester bonds to release carboxylate (EC 3.1.1). It may particularly have lipase activity (triacylglycerol lipase activity, EC 3.1.1.3), i.e. hydrolytic activity for carboxylic ester bonds in triglycerides, e.g. 1,3-specific activity.

Specific Variants

The following are some examples of variants of the T. lanuginosus lipase. Corresponding substitutions may be made by making corresponding amino acid substitutions in other fungal lipolytic enzymes:

D27N
D111G + S216P
L227F
L227F + V228I
G225P
S224I + G225W + T226N + L227P + V228C
S224Y + G225W + T226N + L227P + V228C
D27R + D111G + S216P
D27S + D111G + S216P
D27N + D111A
D27R + D111G + S216P + L227P + P256T
D27R + D111G + S216P + L227G + P256T
D27R + D111G + S216P + L227F + P256T
D27R + D111G + S216P + L227V + P256T
D27R + D111G + S216P + L227G
D27R + D111G + S216P + L227X
027P + D111G + S216P + L227X

Thermostability

The thermostability can be measured at a relevant pH for the intended application using a suitable buffer. Examples of buffers and pH are: pH 10.0 (50 mM glycine buffer), pH 7.0 (50 mM HEPES Buffer) or pH 5.0 (50 mM sodium acetate as buffer).

For comparison, measurements should be made in the same buffer, at the same conditions and at the same protein concentration. Various methods can be used for measuring the thermostability:

Differential Scanning Calorimetry (DSC)

In DSC, the heating rate may be 90 degrees per hour. The sample may be purified to homogeneity, and the melting temperature (T_{M}) may be taken as an expression of the thermostability.

Residual Enzyme Activity

Alternatively, the thermostability can be determined by measuring residual lipolytic enzyme activity after incubation at selected temperatures. p-nitrophenyl ester in 10 mM Tris-HCl, pH 7.5 may be used as the substrate, as described in Giver et al., Proc. Natl. Acad. Sci. USA 95(1998)12809-12813 and Moore et al. Nat. Biotech. 14(1996) 458-467. Samples may be added periodically, or only one sample may be used with or without different additives to prevent or enhance denaturing, e.g. in a 96 well format.

CD Spectroscopy

CD spectroscopy as described e.g. in Yamaguchi et al. Protein engineering 9(1996)789-795. Typical enzyme concentration is around 1 mg/ml, Temperature between 5-80 degrees

Use of Variant

The lipolytic enzyme variants may be used in various processes, and some particular uses are described below. The variant is typically used at 60-95° C. (particularly 75-90° C., 70-90° C. or 70-85° C.) and pH 4.5-11 (particularly 4.5-8 or 5-6.5).

Use in the Paper and Pulp Industry

The lipase may be used in a process for avoiding pitch troubles in a process for the production of mechanical pulp or a paper-making process using mechanical pulp, which comprises adding the lipase to the pulp and incubating. The lipase addition may take place in the so-called white water (recycled process water). It may also be used to remove ink from used paper. The improved thermostability allows the variant to be used at a higher temperature, generally preferred in the industry. This may be done in analogy with WO 9213130, WO 9207138, JP 2160984 A, EP 374700.

Use in Cereal-Based Food Products

The lipolytic enzyme variant may be added to a dough, and the dough may be used to prepare a baked product (particularly bread), pasta or noodles. The improved thermostability of the variant allows it to remain active for a longer time during the heating step (baking, boiling or frying). This may be done in analogy with WO 94/04035, WO 00/32758 , PCT/DK 01/00472, EP 1057415.

The addition of the variant may lead to improved dough stabilization, i.e. a larger loaf volume of the baked product and/or a better shape retention during baking, particularly in a stressed system, e.g. in the case of over-proofing or over-mixing. It may also lead to a lower initial firmness and/or a more uniform and fine crumb, improved crumb structure (finer crumb, thinner cell walls, more rounded cells), of the baked product, and it may further improve dough properties, e.g. a less soft dough, higher elasticity, lower extensibility.

Use in the Fat and Oil Industry

The lipolytic enzyme variant may be used as a catalyst in organic synthesis, e.g. in a process for hydrolyzing, synthesizing or interesterifying an ester, comprising reacting the ester with water, reacting an acid with an alcohol or interesterifying the ester with an acid, an alcohol or a second ester in the presence of the lipolytic enzyme variant. Favorably, the improved thermostability allows the process to be conducted at a relatively high temperature which may be favorable to increase the rate of reaction and to process high-melting substrates.

The ester may be a carboxylic acid ester, e.g. a triglyceride. The interesterification may be done in the presence or absence of a solvent. The enzyme may be used in immobilized form. The process may be conducted in analogy with WO 8802775, U.S. Pat. No. 6,156,548, U.S. Pat. No. 5,776,741, EP 792106, EP 93602, or EP 307154.

Use in Textile Industry

The variant may be used in a process for enzymatic removal of hydrophobic esters from fabrics, which process comprises treating the fabric with an amount of the lipolytic enzyme effective to achieve removal of hydrophobic esters from fabric. The treatment may be done at a temperature of 75° C. or above, e.g. for a period of 1-24 hours. The treatment may be preceded by impregnating the fabric with an aqueous solution of the lipase variant to a liquor pick-up ratio of 50-200%, and may be followed by washing and rinsing to remove the fatty acids.

The process may be conducted in analogy with U.S. Pat. No. 5,578,489 or U.S. Pat. No. 6,077,316.

Use in Detergents

The variant may be used as a detergent additive, e.g. at a concentration (expressed as pure enzyme protein) of 0.001-10 (e.g. 0.01-1) mg per gram of detergent or 0.001-100 (e.g. 0.01-10) mg per liter of wash liquor. This may be done in analogy with WO 97/04079, WO 97/07202, WO 97/41212, WO 98/08939 and WO 97/43375.

Use for Leather

The variants of the invention can also be used in the leather industry in analogy with GB 2233665 or EP 505920.

Nomenclature for Amino Acid Substitutions

The nomenclature used herein for defining amino acid substitutions uses the single-letter code, as described in WO 92/05249.

Thus, D27N indicates substitution of D in position 27 with N. D27N/R indicates a substitution of D27 with N or R. L227X indicates a substitution of L227 with any other amino acid. D27N+D111A indicates a combination of the two substitutions.

Homology and Alignment

For purposes of the present invention, the degree of homology may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45), using GAP with the following settings for polypeptide sequence comparison: GAP creation penalty of 3.0 and GAP extension penalty of 0.1.

In the present invention, corresponding (or homologous) positions in the lipase sequences of Rhizomucor miehei (rhimi), Rhizopus delemar (rhidl), Thermomyces lanuginosa (former; Humicola lanuginosa) (SP400), Penicillium camembertii (Pcl) and Fusarium oxysporum (FoLnp11), are defined by the alignment shown in FIG. 1 of WO 00/32758.

To find the homologous positions in lipase sequences not shown in the alignment, the sequence of interest is aligned to the sequences shown in FIG. 1. The new sequence is aligned to the present alignment in FIG. 1 by using the GAP alignment to the most homologous sequence found by the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

Procedure for Obtaining Thermostable Variants

Variants of a lipolytic enzyme can be obtained by methods known in the art, such as site-directed mutagenesis, random mutagenesis or localized mutagenesis, e.g. as described in WO 9522615 or WO 0032758.

Thermostable variants of a given parent lipolytic enzyme can be obtained by the following standard procedure:

Mutagenesis (error-prone, doped oligo, spiked oligo)

Primary Screening

Identification of more temperature stable mutants

Maintenance (glycerol culture, LB-Amp plates, Mini-Prep)

Streaking out on another assay plate—secondary screening (1 degree higher then primary screening)

DNA Sequencing

Transformation in Aspergillus

Cultivation in 100 ml scale, purification, DSC

Primary Screening Assay

The following assay method is used to screen lipolytic enzyme variants and identify variants with improved thermostability.

E. coli cells harboring variants of a lipolytic enzyme gene are prepared, e.g. by error-prone PCR, random mutagenesis or localized random mutagenesis or by a combination of beneficial mutants and saturation mutagenesis.

The assay is performed with filters on top of a LB agar plate. E. coli cells are grown on cellulose acetate filters supplied with nutrients from the LB agar plate and under the selection pressure of ampicillin supplied with the LB agar. Proteins including the desired enzyme are collected on a nitrocellulose filter between LB agar and cellulose acetate filter. This nitrocellulose filter is incubated in a buffer of desired pH (generally 6.0) and at the desired temperature for 15 minutes (e. g. 78 degrees for the T. lanuginosus lipase). After quenching the filters in ice-water, the residual lipase activity is determined through the cleavage of indole acetate and the subsequent coloration of the reaction product with nitro-blue tetrazolium chloride as described by Kynclova, E et al. (Journal of Molecular Recognition 8 (1995)139-145).

The heat treatment applied is adjusted so that the parent generation is slightly active, approximately 5-10% compared to samples incubated at room temperature. This facilitates the identification of beneficial mutants.

### EXAMPLES

### Example 1

### Expression of Lipase

Plasmid pMT2188

The Aspergillus oryzae expression plasmid pCaHj 483 (WO 98/00529) consists of an expression cassette based on the Aspergillus niger neutral amylase II promoter fused to the Aspergillus nidulans triose phosphate isomerase non translated leader sequence (Pna2/tpi) and the A. niger amyloglycosidase terminater (Tamg). Also present on the plasmid is the Aspergillus selective marker amdS from A. nidulans enabling growth on acetamide as sole nitrogen source. These elements are cloned into the E. coli vector pUC19 (New England Biolabs). The ampicillin resistance marker enabling selection in E. coli of this plasmid was replaced with the URA3 marker of Saccharomyces cerevisiae that can complement a pyrF mutation in E. coli, the replacement was done in the following way:

The pUC19 origin of replication was PCR amplified from pCaHj483 with the primers 142779 (SEQ ID NO: 3) and 142780 (SEQ ID NO: 4).

Primer 142780 introduces a BbuI site in the PCR fragment. The Expand PCR system (Roche Molecular Biochemicals, Basel, Switserland) was used for the amplification following the manufacturers instructions for this and the subsequent PCR amplifications.

The URA3 gene was amplified from the general S. cerevisiae cloning vector pYES2 (Invitrogen corporation, Carlsbad, Calif., USA) using the primers 140288 (SEQ ID 5) and 142778 (SEQ ID 6).

Primer 140288 introduces an EcoRI site in the PCR fragment. The two PCR fragments were fused by mixing them and amplifying using the primers 142780 and 140288 in the splicing by overlap method (Horton et al (1989) Gene, 77, 61-68).

The resulting fragment was digested with EcoRI and BbuI and ligated to the largest fragment of pCaHj 483 digested with the same enzymes. The ligation mixture was used to transform the pyrF E. coli strain DB6507 (ATCC 35673) made competent by the method of Mandel and Higa (Mandel, M. and A. Higa (1970) J. Mol. Biol. 45, 154). Transformants were selected on solid M9 medium (Sambrook et. al (1989) Molecular cloning, a laboratory manual, 2. edition, Cold Spring Harbor Laboratory Press) supplemented with 1 g/l casaminoacids, 500 μg/l thiamine and 10 mg/l kanamycin.

A plasmid from a selected transformant was termed pCaHj 527. ThePna2/tpi promoter present on pCaHj527 was subjected to site directed mutagenises by a simple PCR approach.

Nucleotide 134-144 was altered from SEQ ID NO: 7 to SEQ ID NO: 8 using the mutagenic primer 141223 (SEQ ID NO: 9).

Nucleotide 423-436 was altered from SEQ ID NO: 10 to SEQ ID NO: 11 using the mutagenic primer 141222 (SEQ ID 12).

The resulting plasmid was termed pMT2188.

Plasmid pENI1849

Plasmid pENI1849 was made in order to truncate the pyrG gene to the essential sequences for pyrG expression, in order to decrease the size of the plasmid, thus improving transformation frequency. A PCR fragment (app. 1800 bp) was made using pENI1299 (described in WO 00/24883) as template and the primers 270999J8 (SEQ ID 13) and 270999J9 (SEQ ID 14).

The PCR-fragment was cut with the restriction enzymes StuI and SphI, and cloned into pENI1298 (described in WO 0024883), also cut with StuI and SphI; the cloning was verified by sequencing.

Plasmid pENI1861

Plasmid pENI1861 was made in order to have the state of the art Aspergillus promoter in the expression plasmid, as well as a number of unique restriction sites for cloning.

A PCR fragment (app. 620 bp) was made using pMT2188 (see above) as template and the primers 051199J1 (SEQ ID 15) and 1298TAKA (SEQ ID 16).

The fragment was cut BssHII and Bgl II, and cloned into pEN11849 which was also cut with BssHII and Bgl II. The cloning was verified by sequencing.

Plasmid pENI1902

Plasmid pENI1902 was made in order to have a promoter that works in both E. coli and Aspergillus. This was done by unique site elimination using the “Chameleon double stranded site-directed mutagenesis kit” as recommended by Stratagene®.

Plasmid pENI1861 was used as template and the following primers with 5′ phosphorylation were used as selection primers: 177996 (SEQ ID 17), 135640 (SEQ ID 18) and 135638 (SEQ ID 19).

The 080399J19 primer (SEQ ID NO: 20) with 5′ phosphorylation was used as mutagenic primer to introduce a −35 and −10 promoter consensus sequence (from E. coli) in the Aspergillus expression promoter. Introduction of the mutations was verified by sequencing.

Plasmid pSMin001

Plasmid pSMin001 was made in order to permit the expression of the T. lanuginosus lipase in E. coli and Aspergillus.

Plasmid pAHL (described in WO 9205249) was used as template for PCR to amplify the T. lanuginosus lipase gene with the following Primers: 19671 (SEQ ID NO: 21) and 991213J5 (SEQ ID NO: 22). Primer 991213J5 introduced a SacII site into the PCR fragment. The PCR fragment (appr. 1100 bp) was cut with BamHI and SacII and cloned into pEni1902 cut with the same enzymes. The cloning was verified by DNA sequencing. The plasmid was transformed in E. coli DH5α, and lipase expression was detected by using the described filter assay.

Using this newly developed plasmid it was possible to express the desired enzyme in Aspergillus without any modification. The achieved expression rates in E. coli were quite low, but sufficient for the screening assay.

### Example 2

### Production of Thermostable Lipase Variants

Several techniques were used to create diversity in the T. lanuginosus lipase gene: error-prone PCR, localized random mutagenesis with the aid of doped oligonucleotides, and site-directed mutagenesis.

Variants exhibiting higher temperature stability were selected by the primary assay described above, and were cultivated in LB media and streaked out again on assay plates as described above for a secondary screening. The assay in the secondary screening was performed with a 1-1.5 degrees higher temperature. The DNA of mutants still active under these conditions were sequenced and transformed into Aspergillus to obtain a higher amount of protein, followed by a chromatographic purification. The purified enzyme was used for DSC analysis to prove the enhancement of the stability.

Next, amino acid substitutions found in the beneficial variants were combined, and saturation mutagenesis was used to ensure that all 20 amino acids were introduced in the desired positions.

### Example 3

### Thermostability of Lipase Variants

All samples identified as more thermostable in the primary and secondary screening In Example 2 were purified to homogeneity, and their stability was checked by differential scanning calorimetry (DSC) at pH 5.0 and/or 7.0 to determine the stability of the protein, given by its melting temperature (T_{M}). The parent lipase from T. lanuginosus was included for comparison.

Eight variants were found to have increased thermostability at pH 5.0, four variants showing an increase of more than 4° C. Two variants were tested at pH 7.0 and found to have improved thermostability.

### Example 4

### Thermostability of Lipase Variants by DSC

A number of variants of the T. lanuginosus lipase were prepared and purified, and the thermostability was checked by differential scanning calorimetry (DSC) at pH 5.0 to determine the stability of the protein, given by its melting temperature (T_{M}). The parent lipase from T lanuginosus was included for comparison.

The following variants were found to be more thermostable than the parent lipase:

D111G + S216P
D27N
L227F
S224I + G225W + T226N + L227P + V228C
L227F + V228I
G225P
W221C + G246C

The following variants were found to be more thermostable than the parent lipase with at least 4° C. increase of the melting temperature.

D27R + D111G + S216P
D27N + D111A
D27R + D111G + S216P + L227G + P256T
D27R + D111G + S216P + L227F + P256T
D27R + D111G + S216P + L227G
D27S + D111G + S216P
D27R + D111A + S216P + L227G + P256T
D27R + D111G + S216P + G225P + L227G + P256T
D27R + T37S + D111G + S216P + L227G + P256T
D27R + N39F + D111G + S216P + L227G + P256T
D27R + G38C + D111G + S216P + L227G + P256T
D27R + D111G + S216P + L227G + T2441 + P256T
D27R + G91A + D111G + S216P + L227G + P256T
N25I + D27R + D111A + S216P + L227G + P256T
N25L + D27R + D111A + S216P + L227G + P256T
N26D + D27R + D111A + S216P + L227G + P256T
D27R + K46R + D111A + S216P + L227G + P256T
D27R + V60N + D111A + S216P + L227G + P256T
D27R + D111A + P136A + S216P + L227G + P256T
D27R + D111A + S216P + L227G + P256T + I265F
D27R + S58Y + D111A + S216P + L227G + P256T +
N26D + D27R + E56Q + D111A + S216P + L227G + P256T
D27R + G91A + D96E + L97Q + D111A + S216P + L227G + P256T
D27R + G91A + D111A + S216P + L227G + P256T +
D27R + G91T + N94S + D111A + S216P + L227G + P256T
D27R + G91S + D111A + S216P + L227G + P256T +
D27R + G91N + D111A + S216P + L227G + P256T
D27R + D96E + D111A + S216P + L227G + P256T
D27R + I90L + G91A + N94K + D111A + S216P + L227G + P256T
D27R + G91S + F95V + D111A + S216P + L227G + P256T

### Example 5

### Thermostability by Plate Assay

A number of variants of the T. lanuginosus lipase were prepared and tested for thermostability as described above under “primary screening assay”. The parent lipase from T. lanuginosus was included for comparison.

The following variants were found to be more thermostable than the parent lipase:

D27R + I90V + G91S + D111A + S216P + L227G + P256T
D27R + G91N + N94R + D111A + S216P + L227G + P256T
D27R + I90L + L93F + 096N +
D111A + S216P + L227G + P256T
D27R + I90L + G91A + D96E +
D111A + S216P + L227G + P256T
D27R + G91S + L93F + D111A + S216P + L227G + P256T
D27R + G91T + N94K + D111A + S216P + L227G + P256T
D27R + G91T + 0111A + S216P + L227G + P256T
D27R + L93F + D111A + D137N + S216P + L227G + P256T
D27R + G91S + 096N + D111A + S216P + L227G + P256T
D27R + G91W + D111A + S216P + L227G + P256T
D27R + I90L + G91T + D111A + S216P + L227G + P256T
D27R + G91S + L93F + N94R +
D96G + D111A + S216P + L227G + P256T
D27R + G91T + D96N + D111A + S216P + L227G + P256T
D27R + I90V + G91T + L93F + N94K +
D111A + S216P + L227G + P256T
D27R + L93V + D111A + S216P + L227G + P256T
D27R + G91S + N94K + D111A + S216P + L227G + P256T
D27R + I90L + G91T + D111A + S216P + L227G + P256T
D27R + G91S + L93F + F951 + D96N +
D111A + S216P + L227G + P256T
D27R + D111A + V187I + S216P + L227G + P256T
D27R + D111A + F211Y + S216P + L227G + P256T
D27R + R118M + D111A + A131V + S216P + L227G + P256T
D27R + P29S + R84C + D111A +
H135Y + S216P + L227G + P256T
D27R + T32S + D111A + H135Y + S216P + L227G + P256T
D27R + G91R + D111A + 1238V + S216P + L227G + P256T
D27R + F51I + I76V + N101D + D111A +
N162R + S216P + L227G + P256T
D27R + F51L + D111A + S216P + L227G + P256T

1. A variant of a parent fungal lipolytic enzyme, wherein the variant
a) has an amino acid sequence which compared to the parent lipolytic enzyme comprises substitution of an amino acid residue corresponding to any of amino acids 21, 27, 29, 32, 34-42, 51, 54, 76, 84, 90-97, 101, 105, 111, 118, 125, 131, 135, 137, 162, 187, 189, 206-212, 216, 224-234, 242-252 and 256 of SEQ ID NO: 1, and
b) is more thermostable than the parent lipolytic enzyme.

2. The variant of the preceding claim which is at least 4° C. more thermostable than the parent lipolytic enzyme.

3. The variant of either preceding claim, wherein the amino acid residue is substituted with an amino acid residue different from Pro.

4. The variant of any preceding claim wherein the parent lipolytic enzyme has at least 50% homology with SEQ ID NO: 1.

5. The variant of the preceding claim wherein the parent lipolytic enzyme is the lipase produced by Thermomyces lanuginosus DSM 4109 and having the amino acid sequence of SEQ ID NO: 1.

6. The variant of either preceding claim which comprises substitution of an amino acid residue corresponding to Y21, D27, P29, T32, A40, F51, S54, I76, R84, I90, G91, N94, N101, S105, D111, R118, R125, A131, H135, D137, N162, V187, T189, E210, G212, S216, G225, L227, I238 or P256 of SEQ ID NO:1.

7. The variant of any preceding claim which comprises one or more substitutions corresponding to D27N/R/S, P29S, T32S, F51I/L, I76V, R84C, I90L/N, G91A/N/S/T/W, L93F, N94K/R/S, F95I, D96G/N, N101D, D111A/G, R118M, A131V, H135Y, D137N, N162R, V187I, F211Y, S216P, S224I/Y, G225P, T226N, L227F/P/G/V, L227X, V228C/I, 238V and P256T of SEQ ID NO: 1.

8. The variant of any preceding claim which has one, two, three, four, five, six, seven or eight of said substitutions.

9. The variant of any preceding claim which further comprises one or more substitutions of amino acid residues other than those listed in claim 1, preferably 1-5 such substitutions.

10. The variant of any preceding claim which comprises substitutions corresponding to the following in SEQ ID NO: 1:
a) D27N
b) D111G+S216P
c) L227F
d) L227F+V228I
e) G225P
f) S224I+G225W+T226N+L227P+V228C
g) S224Y+G225W+T226N+L227P+V228C
h) D27R+D111G+S216P
i) D27S+D111G+S216P
j) D27N+D111A
k) D27R+D111G+S216P+L227P+P256T
l) D27R+D111G+S216P+L227G+P256T
m) D27R+D111G+S216P+L227F+P256T
n) D27R+D111G+S216P+L227V+P256T
o) D27R+D111G+S216P+L227G
p) D27R+D111G+S216P+L227X
q) D27P+D111G+S216P+L227X
r) S224I+G225W+T226N+L227P+V228C
s) W221C+G246C
t) D27R+D111G+S216P
u) D27N+D111A
v) D27R+D111G+S216P+L227G+P256T
w) D27R+D111G+S216P+L227F+P256T
x) D27R+D111G+S216P+L227G
y) D27S+D111G+S216P
z) D27R+D111A+S216P+L227G+P256T
aa) D27R+D111G+S216P+G225P+L227G+P256T
bb) D27R+T37S+D111G+S216P+L227G+P256T
cc) D27R+N39F+D111G+S216P+L227G+P256T
dd) D27R+G38C+D111G+S216P+L227G+P256T
ee) D27R+D111G+S216P+L227G+T2441+P256T
ff) D27R+G91A+D111G+S216P+L227G+P256T
gg) N25I+D27R+D111A+S216P+L227G+P256T
hh) N25L+D27R+D111A+S216P+L227G+P256T
ii) N26D+D27R+D111A+S216P+L227G+P256T
jj) D27R+K46R+D111A+S216P+L227G+P256T
kk) D27R+V60N+D111A+S216P+L227G+P256T
ll) D27R+D111A+P136A+S216P+L227G+P256T
mm) D27R+D111A+S216P+L227G+P256T+I265F
nn) D27R+S58Y+D111A+S216P+L227G+P256T+
oo) N26D+D27R+E56Q+D111A+S216P+L227G+P256T
pp) D27R+G91A+D96E+L97Q+D111A+S216P+L227G+P256T
qq) D27R+G91A+D111A+S216P+L227G+P256T+
rr) D27R+G91T+N94S+D111A+S216P+L227G+P256T
ss) D27R+G91S+D111A+S216P+L227G+P256T+
tt) D27R+G91N+D111A+S216P+L227G+P256T
uu) D27R+D96E+D111A+S216P+L227G+P256T
vv) D27R+I90L+G91A+N94K+D111A+S216P+L227G+P256T
ww) D27R+G91S+F95V+D111A+S216P+L227G+P256T

11. The variant of any preceding claim having a denaturation temperature which is at least 5° C. higher than the parent lipolytic enzyme, preferably measured at pH 5-7.

12. A DNA sequence encoding the variant of any preceding claim.

13. A vector comprising the DNA sequence of the preceding claim.

14. A transformed host cell harboring the DNA sequence of claim 12 or the vector of claim 13.

15. A method of producing the variant of any of claims 1-11 comprising
a) cultivating the cell of claim 14 so as to express and preferably secrete the variant, and
b) recovering the variant.

16. A method of producing a lipolytic enzyme variant comprising:
a) selecting a parent fungal lipolytic enzyme,
b) in the parent lipolytic enzyme substituting at least one amino acid residue corresponding to any of 21, 27, 29, 32, 34-42, 51, 54, 76, 84, 90-97, 101, 105, 111, 118, 125, 131, 135, 137, 162, 187, 189, 206-212, 216, 224-234, 242-252 and 256 of SEQ ID NO: 1,
c) optionally, substituting one or more amino acids other than b),
d) preparing the variant resulting from steps a)-c),
e) testing the thermostability of the variant,
f) selecting a variant having an increased thermostability, and
g) producing the selected variant.

17. The method of the preceding claim wherein the parent lipolytic enzyme has at least 50% homology with SEQ ID NO: 1.

18. The method of the preceding claim wherein the parent lipolytic enzyme is the lipase produced by Thermomyces lanuginosus DSM 4109 and having the amino acid sequence of SEQ ID NO: 1.

19. The method of either preceding claim which comprises substituting an amino acid residue corresponding to Y21, D27, P29, T32, A40, F51, S54, I76, R84, I90, G91, N94, N101, S105, D111, R118, R125, A131, H135, D137, N162, V187, T189, E210, G212, S216, G225, L227, I238 or P256 of SEQ ID NO:1.

20. The method of the preceding claim which comprises substituting an amino acid residue corresponding to D27N/R/S, P29S, T32S, F51I/L, I76V, R84C, I90L/V, G91A/N/S/T/W, L93F, N94K/R/S, F95I, D96G/N, N101D, D111A/G, R118M, A131V, H135Y, D137N, N162R, V187I, F211Y, S216P, S224I/Y, G225P, T226N, L227F/P/G/V, L227X, V228C/I, 238V and P256T of SEQ ID NO: 1.

21. A process for hydrolyzing a carboxylic acid ester, comprising incubating the ester with the lipase of any of claims 1-11 in the presence of water.

22. A process for controlling pitch troubles in a process for the production of mechanical pulp or a paper-making process using mechanical pulp, which comprises adding the lipase of any of claims 1-11 to the pulp and incubating.

23. The process of either preceding claim wherein the incubation is done at a temperature 60-95° C., particularly 75-90° C.

24. The process of any preceding claim, wherein the incubation is done at a pH in the range 4.5-11, particularly 5-6.5.

25. A process for preparing a dough or a baked product prepared from the dough, comprising adding the lipolytic enzyme of any of claims 1-11 to the dough.

26. A process for hydrolyzing, synthesizing or interesterifying an ester, comprising reacting the ester with water, reacting an acid with an alcohol or interesterifying the ester with an acid, an alcohol or a second ester in the presence of the lipolytic enzyme of any of claims 1-11.

27. A process for enzymatic removal of hydrophobic esters from fabrics, which process comprises treating the fabric with an amount of the lipolytic enzyme of any of claims 1-11 effective to achieve removal of hydrophobic esters from fabric.
